(19) United States
(12) Patent Application Publication
(10) Pub. No.: US 2014/0262743 A1
(43) Pub. Date: Sep. 18, 2014

(54) System for Harvesting Oriented Light for Water Splitting and Carbon Dioxide Reduction

(71) Applicant: Sunpower Technologies LLC, (US)

(72) Inventors: Daniel Landry, Redondo Beach, CA (US); Travis Jennings, San Diego, CA (US)

(73) Assignee: Sunpower Technologies LLC, San Marcos, CA (US)

(21) Appl. No.: 13/801,169

(22) Filed: Mar. 13, 2013

(51) Int. Cl. B01J 19/12; C07C 1/12

(52) U.S. Cl. CPC B01J 19/127; C07C 1/12
USPC 204/157.15; 422/162

(57) ABSTRACT

A photosynthetic system for splitting water to produce hydrogen and using the produced hydrogen for the reduction of carbon dioxide into methane is disclosed. The disclosed photosynthetic system employs photoactive materials that include oriented photocatalytic capped colloidal nanocrystals (PCCN) within their composition, in order to harvest sunlight and obtain the energy necessary for water splitting and subsequent carbon dioxide reduction processes. The photosynthetic system may also include elements necessary to transfer water produced in the carbon dioxide reduction process, for subsequent use in water splitting process. The systems may also include elements necessary to store oxygen and collect and transfer methane for subsequent transformation of methane into energy.

### CROSS-REFERENCE TO RELATED APPLICATIONS

The disclosure here described is related to the invention disclosed in the U.S. application Ser. No. (not yet assigned), entitled “Photocatalyst for the Production of Hydrogen” and U.S. application Ser. No. (not yet assigned), entitled “Artificial Photosynthetic System using Photocatalyst”.

### BACKGROUND

1. Technical Field

The present disclosure relates generally to artificial photosynthetic systems, in particular to a system that combines oriented photocatalyst semiconductor surfaces with hydrogen and methane production systems.

2. Background Information

The prior art describes the formation of photocatalytic nanoparticles in various classical polymers, such as organization and immobilization of metal compounds in linear, branched, and cross-linked polymers.

In general, current photocatalytic systems, employing random oriented nanocrystals, suffer from low reaction rates. Reaction-induced changes in pH, donor concentrations, orientation and surface trap sites are at least partly responsible for low reaction rates observed.

There is a need for an optimization of complete photosynthetic systems that may be used for light harvesting and converting water and carbon dioxide (CO₂) into methane fuel, using photocatalytic semiconductors with the ability to improve the efficiency of a photosynthetic system in order to make it commercially viable.

### SUMMARY

The present disclosure refers to an artificial photosynthetic system employing sunlight. This system may include a first photoactive material to split water into hydrogen and oxygen, for subsequent use of hydrogen in the same artificial photosynthetic system with a second photoactive material for carbon dioxide reduction into water and methane. Reflective or polarizing surfaces may be employed to collect solar energy and orient light rays for maximum absorption and energy conversion with oriented photocatalytic surfaces.

Photoactive materials in the present disclosure may include oriented photocatalytic capped colloidal nanocrystals (PCCN) structured with semiconductor nanocrystals, exhibiting the ability to absorb light for producing charge carriers to accelerate necessary redox reactions and prevent charge carriers recombination.

In another aspect of the disclosure, the PCCN composition may be deposited on a substrate as thin or bulk films by a variety of techniques known in the art, producing short or long range ordering of PCCN. Subsequently, an application of orientational methods known in the art may be applied to the photoactive material. Additionally, the deposited PCCN composition may be thermally treated to anneal and form inorganic matrices with embedded PCCN. In another aspect of the disclosure, a light polarizing system may be included. The system configuration may change depending on the final user needs.

The artificial photosynthetic system may include the splitting of water into hydrogen and oxygen, for which a continuous flow of water may enter a first reaction vessel and may subsequently pass through a region including the first photoactive material. When light makes contact with semiconductor nanocrystals, charge separation may occur. Consequently, hydrogen molecules in water may be reduced. Semiconductor nanocrystals in first photoactive material may absorb light at different tunable wavelengths as a function of the particle size and, generally, at shorter wavelengths from the bulk material.

After first reaction vessel, hydrogen and oxygen may migrate through an opening into a gas collecting chamber, which may include a suitable permeable membrane to transfer hydrogen to a second reaction vessel. Gas collecting chamber may include a suitable permeable membrane to transfer oxygen and collect it in a storage tank.

Similarly, carbon dioxide may be injected to the second reaction vessel. According to embodiments, photocatalytic system disclosed may employ CO₂, produced as a byproduct during manufacturing processes, such as carbon dioxide coming from a boiler or other combustion equipment. Hydrogen, transferred from gas collecting chamber, and carbon dioxide may pass through a second photoactive material prior to entering the second reaction vessel.

When light with energy higher than that of the band gap of semiconductor nanocrystals within second photoactive material makes contact with second photoactive material, the process of charge separation may take place. Consequently, electrons from photoactive material may reduce carbon dioxide into water and methane through a series of reactions.

The structure of the inorganic capping agents within both photoactive materials may speed up redox reactions by quickly transferring charge carriers sent by semiconductor nanocrystals to water in order that the consequent water splitting and CO₂ reduction may take place at a faster and more efficient rate and at the same time inhibiting electron-hole recombination.

Any suitable light source may be employed to provide light for both water splitting and CO₂ reduction. A preferable light source may be sunlight, including infrared light which may be used to heat water and also including ultraviolet light and visible light.

Artificial photosynthetic systems, according to embodiments, may be mounted on a structure such as the roof of a building, or may be free standing, such as in a field.

Oriented semiconductor nanocrystals in the oriented photoactive material may absorb the linearly polarized light at different tunable wavelengths as a function of the particle size and generally at shorter wavelengths from the bulk material. Materials of the semiconductor nanocrystals may be selected in accordance with the irradiation wavelength. According to various embodiments, PCCN may exhibit a plurality of suitable configurations, including sphere, tetrapod, and core/shell, among others. The structure of the inorganic capping agents may speed up the reaction by quickly transferring charge carriers sent by semiconductor nanocrystals to water and CO₂, so that the redox reaction and consequent water splitting and CO₂ reduction take place at a faster and more efficient rate and at the same time inhibiting electron-hole recombination. As a result of employing the oriented photoactive material of the present disclosure in combination with a light polarization system, greater sunlight energy extraction may be achieved. In addition, semiconductor nanocrystals may provide for higher surface area available for the absorption of light.

In one embodiment, a method for water splitting and carbon dioxide reduction comprises: forming photocatalytic capped colloidal nanocrystals, wherein each photocatalytic capped colloidal nanocrystal includes a first semiconductor nanocrystal capped with a first inorganic capping agent; depositing the formed photocatalytic capped colloidal nanocrystals onto a first substrate and a second substrate, thereby creating first and second photoactive materials; orienting the photocatalytic capped colloidal nanocrystals of the first photoactive material; orienting the photocatalytic capped colloidal nanocrystals of the second photoactive material; absorbing irradiated light with an energy equal to or greater than the band gap of the semiconductor nanocrystals by the first photoactive material to create charge carriers in a conduction band and holes in a valence band of the photocatalytic capped colloidal nanocrystals of the first photoactive material; passing water through a first reaction vessel so that the water reacts with the first photoactive material to form hydrogen and oxygen, wherein the charge carriers in the conduction band reduce hydrogen molecules from the water and the holes in the valence band oxidize oxygen molecules from the water; separating the hydrogen from the oxygen using a hydrogen permeable membrane and an oxygen permeable membrane; passing the separated hydrogen from the first reaction vessel into a second reaction vessel; passing carbon dioxide into the second reaction vessel; absorbing irradiated light with an energy equal to or greater than the band gap of the semiconductor nanocrystals by the second photoactive material to create charge carriers in a conduction band and holes in a valence band of the photocatalytic capped colloidal nanocrystals of the second photoactive material; reacting the carbon dioxide and the hydrogen with the second photoactive material in the second reaction vessel so that the charge carriers in the conduction band reduce carbon dioxide into methane and the holes in the valence band oxidize the hydrogen into water vapor; and collecting the methane using a methane permeable membrane.

In another embodiment, a method for water splitting and carbon dioxide reduction comprises: absorbing irradiated light with an energy equal to or greater than the band gap of semiconductor nanocrystals in a first photoactive material to create charge carriers in a conduction band and holes in a valence band of photocatalytic capped colloidal nanocrystals of the first photoactive material; passing water through a first reaction vessel so that the water reacts with the first photoactive material to form hydrogen and oxygen, wherein the charge carriers in the conduction band reduce hydrogen molecules from the water and the holes in the valence band oxidize oxygen molecules from the water; separating the hydrogen from the oxygen using a hydrogen permeable membrane and an oxygen permeable membrane; collecting the separated oxygen in an oxygen storage tank; passing the separated hydrogen from the first reaction vessel into a second reaction vessel; transferring carbon dioxide into the second reaction vessel from boiler that produces carbon dioxide through a combustion reaction; absorbing irradiated light with an energy equal to or greater than the band gap of semiconductor nanocrystals in a second photoactive material to create charge carriers in a conduction band and holes in a valence band of photocatalytic capped colloidal nanocrystals of the second photoactive material; reacting the carbon dioxide and the hydrogen with the second photoactive material in the second reaction vessel so that the charge carriers in the conduction band reduce carbon dioxide into methane and the holes in the valence band oxidize the hydrogen into water vapor; separating the methane using a methane permeable membrane; collecting the separated methane in a storage tank; and recycling the water vapor to the first reaction vessel.

In another embodiment, a photosynthetic system comprises: first and second oriented photoactive materials, wherein the first and second oriented photoactive materials include oriented photocatalytic capped colloidal nanocrystals; a first reaction vessel housing the first oriented photoactive material and configured to receive water through an inlet and facilitate a water splitting reaction that produces hydrogen and oxygen when the water reacts with the photocatalytic capped colloidal nanocrystals, wherein the water splitting reaction occurs when the photocatalytic capped colloidal nanocrystals absorb irradiated light to separate charge carriers of the first oriented photoactive material; and a second reaction vessel housing the second oriented photoactive material and configured to receive carbon dioxide through a first inlet, receive hydrogen from the first reaction vessel, and facilitate a carbon dioxide reduction reaction and a hydrogen oxidization reaction that produces methane and water vapor, wherein the reaction begins when the photocatalytic capped colloidal nanocrystals of the second photoactive material absorb polarized light to separate charge carriers of the second oriented photoactive material.

Numerous other aspects, features of the present disclosure may be made apparent from the following detailed description, taken together with the drawing figures.

Additional features and advantages of an embodiment will be set forth in the description which follows, and in part will be apparent from the description. The objectives and other advantages of the invention will be realized and attained by the structure particularly pointed out in the exemplary embodiments in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

Embodiments of the present invention are described by way of example with reference to the accompanying figures, which are schematic and are not intended to be drawn to scale. Unless indicated as representing the prior art, the figures represent aspects of the invention.

FIG. 1 is a block diagram of a method for forming a composition of PCCN, according to an embodiment.

FIG. 2 depicts a PCCN in nanorod configuration, according to an embodiment.

FIG. 3 illustrates a transition dipole moment characterization within PCCN, according to an embodiment

FIG. 4 is a flowchart of a method for forming oriented photocatalyst semiconductor surfaces, according to an embodiment.

FIG. 5 depicts an alignment process employing electric fields, according to an embodiment.

FIG. 6 depicts oriented PCCN in nanorod configuration showing oriented dipole moment receiving light, according to an embodiment.

FIG. 7 illustrates oriented PCCN in nanorod configuration upon a substrate, forming oriented photoactive material employed in the present disclosure, according to an embodiment.

FIG. 8 depicts a charge separation process, according to an embodiment.

FIG. 9 shows a light polarization method, according to an embodiment.

FIG. 10 shows a multiple mirror configuration, according to an embodiment.

FIG. 11 illustrates a focusing mirrors configuration, according to an embodiment.

FIG. 12 shows a photosynthetic system, according to an embodiment.

### DETAILED DESCRIPTION

Disclosed here is a photosynthetic system employing PCCN that may be included in a photoactive material where methane and water are produced by a carbon dioxide reduction process in the presence of hydrogen obtained from a water splitting process, according to an embodiment.

In the following detailed description, reference is made to the accompanying drawings, which form a part hereof. In the drawings, which are not to scale or to proportion, similar symbols typically identify similar components, unless context dictates otherwise. The illustrative embodiments described in the detailed description, drawings and claims, are not meant to be limiting. Other embodiments may be used and/or and other changes may be made without departing from the spirit or scope of the present disclosure.

### DEFINITIONS

As used here, the following terms may have the following definitions:

“Alignment ligand” refers to components that interact with one or more nanostructures and can be used to order, orient and/or align the nanostructures associated therewith

“Electron-hole pairs” refers to charge carriers that are created when an electron acquires energy sufficient to move from a valence band to a conduction band and creates a free hole in the valence band, thus starting a process of charge separation.

“Electric dipole moment” refers to the separation of positive and negative charge on a system.

“Inorganic capping agent” refers to semiconductor particles that cap semiconductor nanocrystals.

“Orientation” refers to the rotation needed to bring a nanocrystal into position or alignment so that its longitudinal axis has a desired angle.

“Photoactive material” refers to at least one substance that may be used in photocatalytic processes for absorbing light and starting a chemical reaction with light.

“Polarization” refers to a process in which waves of light are restricted to certain directions of vibration.

“Semiconductor nanocrystals” refers to particles sized between about 1 and about 100 nanometers produced using semiconducting materials with high surface areas able to absorb light.

“Transition dipole moment” refers to the axis of a system that may interact with light of a certain polarization.

### DESCRIPTION OF DRAWINGS

Method for Growing Oriented Semiconductor Nanocrystals

Controlling the orientation of the semiconductor nanocrystals in a substrate may allow controlling different parts of the light spectrum in the same system, therefore, increasing the efficiency in the light harvesting process. A homogeneous orientation of the nanocrystals upon a substrate may be achieved employing a variety of state of the art methods, such as template-driven seeded growth, electric fields application, or other appropriate orientational forces. The orientation of the nanocrystals may be along either 1 crystallographic axis (1D orientation), or orientation along 2 axes (2D orientation). Once orientation is fixed along 2 axes, the 3rd axis may be already fixed for a rigid structure.

In an embodiment, semiconductor nanocrystals may be grown employing a known in the art method for template-driven seeded growth. Seeded growth refers to methods for growing crystals in which a seed crystal may be used to initiate crystal lattice growth and elongation (as opposed to forcing a nucleation event before crystal growth may be observed). In an embodiment, the seed crystal may be freely dispersed in a solution, or may be deposited on a substrate. In another embodiment, the seed crystal may be the substrate itself, or may be composed of the same material as the intended semiconductor nanocrystal. In another embodiment, the seed crystal may be composed of another crystalline material with the proper crystal lattice structure, atomic spacing, and surface energy to promote further crystal growth. For example, GaSb has shown to be an appropriate surface for semiconductor growth. Accordingly, a GaSb single nanocrystal surface may be used to seed the growth of a semiconductor nanocrystal using molecular beam epitaxy (MBE), or chemical beam epitaxy (CBE) so that nanocrystal growth may be templated by the substrate crystal structure. Photocatalyst layers would then be grown on top of the aligned and oriented semiconductor nanocrystal.

The seeded growth method may have the benefits of lowering the activation energy required for crystal growth to occur, as well as other reaction parameters, such as monomer concentration and reaction temperature; and allowing a degree of control over deposition density, growth rate, and orientation dispersion to yield a highly uniform and oriented nanocrystal surface with 2D/3D orientation.

The morphologies of semiconductor nanocrystals may include nanorods, nanoplates, nanowires, dumbbell-like nanoparticles, and dendritic nanomaterials, among others. Each morphology may include an additional variety of shapes such as spheres, cubes, tetrahedra (tetrapods), among others.

To modify optical properties as well as to enhance charge carriers mobility, semiconductor nanocrystals may be capped by inorganic capping agents in polar solvents instead of organic capping agents. In those embodiments, inorganic capping agents may act as photocatalysts to facilitate a photocatalytic reaction on the surface of semiconductor nanocrystals. Optionally, semiconductor nanocrystals may be modified by the addition of not one but two different inorganic capping agents. In that instance, a reduction inorganic capping agent may be first employed to facilitate the reduction half-cell reaction; then, an oxidation inorganic capping agent facilitates the oxidation half-cell reaction. Inorganic capping agents may be neutral or ionic, or they may be discrete species, either linear or branched chains, or two-dimensional sheets. Ionic inorganic capping agents are commonly referred to as salts, pairing a cation and an anion. The portion of the salt specifically referred to as an inorganic capping agent is the ion that displaces the organic capping agent.

Method for Forming Composition of Photocatalytic Capped Colloidal Nanocrystals (PCCN)

FIG. 1 shows a flow diagram of a method 100 for forming a composition of PCCN 102, according to an embodiment. PCCN 102 may be synthesized following accepted protocols, and may include one or more semiconductor nanocrystals 104 and one or more inorganic capping agents.

Method 100 for forming a composition of PCCN 102 may include a first step where semiconductor nanocrystals 104 may be grown by reacting as semiconductor nanocrystal 104 precursors in the presence of an organic solvent, here referred to as organic capping agent, by an addition of the organic capping agent 106. Additionally, the long organic chains radiating from organic capping agents on the surface of semiconductor nanocrystal 104 precursors may assist in the suspension and/or solubility of semiconductor nanocrystal 104 precursors in a solvent. The chemistry of capping agents may control several system parameters, for example, the size of semiconductor nanocrystal 104 precursors, growth rate or shape, the dispersability in various solvents and solids, and even the excited state lifetimes of charge carriers in semiconductor nanocrystal 104 precursors. The flexibility of synthesis is demonstrated by the fact that often one capping agent may be chosen for its growth control properties, and then later a different capping agent may be substituted to provide a more suitable interface or to modify optical properties or charge carrier mobility.

For the substitution of organic capping agents with inorganic capping agents, organic capped semiconductor nanocrystals 104 in the form of a powder, suspension, or a colloidal solution, may be mixed by an addition of inorganic capping agents 108, causing a reaction of organic capped semiconductor nanocrystals 104 with inorganic capping agents. This reaction rapidly produces insoluble and intractable materials. Afterwards, an addition of immiscible solvents 110 may be made causing the dissolution of organic capping agents and inorganic capping agents 112. These two solutions may then be mixed 114, by combining and stirring them for about 10 minutes, after which a complete transfer of organic capped semiconductor nanocrystals 104 from the non-polar solvent to the polar solvent may be observed. During this exchange, organic capping agents are released. Generally, inorganic capping agents may be dissolved in a polar solvent, while organic capped semiconductor nanocrystals 104 may be dissolved in an immiscible, generally non-polar, solvent. Addition of immiscible solvents 110 may control the reaction, facilitating a rapid and complete replacement of organic capping agents with inorganic capping agents 116

Organic capped semiconductor nanocrystals 104 may react with inorganic capping agents at or near the solvent boundary, where a portion of the organic capping agent may be exchanged/replaced with a portion of the inorganic capping agent. Thus, inorganic capping agents may displace organic capping agents from the surface of semiconductor nanocrystal 104 precursors, and inorganic capping agents may bind to that semiconductor nanocrystal surface. This process may continue until equilibrium is established between inorganic capping agents and the free inorganic capping agents. Preferably, the equilibrium favors inorganic capping agents. All the steps described above may be carried out in a nitrogen environment inside a glove box.

Subsequently, an isolation procedure, such as the precipitation of inorganic product, may be required for the purification of inorganic capped semiconductor nanocrystals 118 to form a PCCN 102. That precipitation permits one of ordinary skill to wash impurities and/or unreacted materials out of the precipitate. Such isolation may allow for the selective application of PCCN 102.

Neither the morphology nor the size of semiconductor nanocrystal 104 precursors inhibits a method 100 for forming composition of PCCN 102 using the semiconductor nanocrystal 104 precursors; rather, the selection of morphology and size of semiconductor nanocrystal 104 precursors may permit the tuning and control of the properties of PCCN 102.

Examples of semiconductor nanocrystal 104 precursors may include the following: Ag, Au, Ru, Rh, Pt, Pd, Os, Ir, Ni, Cu, CdS, Pt-tipped, TiO₂, Mn/ZnO, ZnO, CdSe, SiO₂, ZrO₂, SnO₂, WO₃, MoO₃, CeO₂, ZnS, WS₂, MoS₂, SiC, GaP, Cu—Au, Ag, and mixtures thereof; Cu/TiO2, Ag/TiO₂, Cu—Fe/TiO₂—SiO₂ and dye-sensitized Cu—Fe/P25 coated optical fibers, AlN, AlP, AlAs, Bi, Bi₂S₃, Bi₂Se₃, Bi₂Te₃, CdS, CdSe, CdTe, Co, CoPt, CoPt₃, Cu₂S, Cu₂Se, CuInSe₂, Culn₍₁₋ₓ₎Gaₓ(S,Se)₂, Cu₂ZnSn(S,Se)₄, Fe, FeO, Fe₂O₃, Fe₃O₄, FePt, GaN, GaP, GaAs, GaSb, GaSe, Ge, HgS, HgSe, HgTe, InN, InP, InSb, InAs, Ni, PbS, PbSe, PbTe, Si, Sn, ZnSe, ZnTe, and mixtures thereof. Examples of applicable semiconductor nanocrystals 104 may include core/shell semiconductor nanocrystals like Au/PbS, Au/PbSe, Au/PbTe, Ag/PbS, Ag/PbSe, Ag/PbTe, Pt/PbS, Pt/PbSe, Pt/PbTe, Au/CdS, Au/CdSe, Au/CdTe, Ag/CdS, Ag/CdSe, Ag/CdTe, Pt/CdS, Pt/CdSe, Pt/CdTe, Au/FeO, Au/Fe₂O₃, Au/Fe₃O₄, Pt/FeO, Pt/Fe₂O₃, Pt/Fe₃O₄, FePt/PbS, FePt/PbSe, FePt/PbTe, FePt/CdS, FePt/CdSe, FePt/CdTe, CdSe/CdS, CdSe/ZnS, InP/CdSe, InP/ZnS, InP/ZnSe, InAs/CdSe, and InAs/ZnSe; nanorods like CdSe, core/shell nanorods like CdSe/CdS; nano-tetrapods like CdTe, and core/shell nano-tetrapods like CdSe/CdS.

The organic solvent may be a stabilizing organic ligand. One example of an organic capping agent may be trioctylphosphine oxide (TOPO). TOPO 99% may be obtained from Sigma-Aldrich Co. LLC (St. Louis, Mo.). TOPO capping agent prevents the agglomeration of semiconductor nanocrystals 104 during and after their synthesis. Other suitable organic capping agents may include long-chain aliphatic amines, long-chain aliphatic phosphines, long-chain aliphatic carboxylic acids, long-chain aliphatic phosphonic acids and mixtures thereof.

Some examples of polar solvents may include 1,3-butanediol, acetonitrile, ammonia, benzonitrile, butanol, dimethylacetamide, dimethylamine, dimethylethylenediamine, dimethylformamide, dimethylsulfoxide (DMSO), dioxane, ethanol, ethanolamine, ethylenediamine, ethyleneglycol, formamide (FA), glycerol, methanol, methoxyethanol, methylamine, methylformamide, methylpyrrolidinone, pyridine, tetramethylethylenediamine, triethylamine, trimethylamine, trimethylethylenediamine, water, and mixtures thereof. Polar solvents like FA, spectroscopy grade, and DMSO, anhydrous, 99.9% may be supplied by Sigma-Aldrich Co. LLC. Suitable colloidal stability of the dispersions of semiconductor nanocrystal 104 precursors is mainly determined by a solvent dielectric constant, which may range between about 106 to about 47, with about 106 being preferred.

Examples of non-polar or organic solvents may include tertiary-Butanol, pentane, pentanes, cyclopentane, hexane, hexanes, cyclohexane, heptane, octane, isooctane, nonane, decane, dodecane, hexadecane, benzene, 2,2,4-trimethylpentane, toluene, petroleum ether, ethyl acetate, diisopropyl ether, diethyl ether, carbon tetrachloride, carbon disulfide, and mixtures thereof. Other examples may include alcohol, hexadecylamine (HDA), hydrocarbon solvents at high temperatures.

Preferred inorganic capping agents for PCCN 102 may include chalcogenides, and zintl ions (homopolyatomic anions and heteropolyatomic anions that may have intermetallic bonds between the same or different metals of the main group, transition metals, lanthanides, and/or actinides, for example, As₃³⁻, As₄²⁻, As₅³⁻, As₇³⁻, Ae₁₁³⁻, AsS₃³⁻, As₂Se₆³⁻, As₂Te₆³⁻, As₁₀Te₃²⁻, Au₂Te₄²⁻, Au₃Te₄³⁻, Bi₃³⁻, Bi₄²⁻, Bi₅³⁻, GaTe²⁻, Ge₉²⁻, Ge₉⁴⁻, Ge₂S₆⁴⁻, HgSe₂²⁻, Hg₃Se₄²⁻, In₂Se₄²⁻, In₂Te₄²⁻, Ni₅Sb₁₇⁴⁻, Pb₅²⁻, Pb₇⁴⁻, Pb₉⁴⁻, Pb₂Sb₂²⁻, Sb₃³⁻Sb₄²⁻, Sb₇³⁻, SbSe₄³⁻, SbSe₄⁵⁻, SbTe₄⁵⁻, Sb₂Se₃⁻, Sb₂Te₅⁴⁻, Sb₂Te₇⁴⁻, Sb₄Te₄⁴⁻, Sb₉Te₆³⁻, Se₂²⁻, Se₃²⁻, Se₄²⁻, Se_{5,6}²⁻, Se₆²⁻, Sn₅²⁻, Sn₉³⁻, Sn₉⁴⁻, SnS₄⁴⁻, SnSe₄⁴⁻, SnTe₄⁴⁻, Sn S₄Mn₂⁵⁻, SnS₂S₆⁴⁻, Sn₂Se₆⁴⁻, Sn₂Te₆⁴⁻, Sn₂Bi₂²⁻, Sn₈Sb³⁻, Te₂²⁻, Te₃²⁻, Te₄²⁻, Tl₂Te₂²⁻, TlSn₈³⁻, TlSn₈⁵⁻, TlSn₉³⁻, TlTe₂²⁻, mixed metal SnS₄Mn₂⁵⁻, and the like), where zintl ions refers to homopolyatomic anions and heteropolyatomic anions that have intermetallic bonds between the same or different metals of the main group, lanthanides, and/or actinides, transition metal chalcogenides, such as, tetrasulfides and tetraselenides of vanadium, niobium, tantalum, molybdenum, tungsten, and rhenium, and the tetratellurides of niobium, tantalum, and tungsten. These transition metal chalcogenides may further include the monometallic and polymetallic polysulfides, polyselenides, and mixtures thereof, e.g., MoS(Se₄)₂²⁻, Mo₂S₆²⁻, and the like, polyoxometalates and oxometalates, such as tungsten oxide, iron oxide, zinc oxide, cadmium oxide, zinc sulfide, gallium zinc nitride oxide, bismuth vanadium oxide, zinc oxide, and titanium dioxide, among others; metals selected from transition metals; positively charged counter ions, such as alkali metal ions, ammonium, hydrazinium, tetraalkylammonium, and the like.

Further embodiments may include other inorganic capping agents. For example, inorganic capping agents may include molecular compounds derived from CuInSe₂, CulnₓGa₁₋ₓSe₂, Ga₂Se₃, In₂Se₃, In₂Te₃, Sb₂S₃, Sb₂Se₃, Sb₂Te₃, and ZnTe.

Method 100 may be adapted to produce a wide variety of PCCN 102. Adaptations of method 100 may include adding two different inorganic capping agents to a single semiconductor nanocrystal 104 precursor, adding two different semiconductor nanocrystal 104 precursors to a single inorganic capping agent, adding two different semiconductor nanocrystal 104 precursors to two different inorganic capping agents, and/or additional multiplicities. The sequential addition of inorganic capping agents 108 to semiconductor nanocrystal 104 precursors may be possible under the disclosed method 100. Depending, for example, upon concentration, nucleophilicity, bond strength between capping agents and semiconductor nanocrystal 104 precursor, and bond strength between semiconductor nanocrystal 104 precursor face dependent capping agent and semiconductor nanocrystal 104 precursor, inorganic capping of semiconductor nanocrystal 104 precursor may be manipulated to yield other combinations.

Suitable PCCN 102 may include ZnS.TiO₂, TiO₂.CuO, ZnS.RuOₓ, ZnS.ReOₓ, Au.AsS₃, Au.Sn₂S₆, Au.SnS₄, Au.Sn₂Se₆, Au.In₂Se₄, Bi₂S₃.Sb₂Te₅, Bi₂S₃.Sb₂Te₇, Bi₂Se₃.Sb₂Te₅, Bi₂Se₃.Sb₂Te₇, CdSe.Sn₂S₆, CdSe.Sn₂Te₆, CdSe.In₂Se₄, CdSe.Ge₂S₆, CdSe.Ge₂Se₃, CdSe.HgSe₂, CdSe.ZnTe, CdSe.Sb₂S₃, CdSe.SbSe₄, CdSe.Sb₂Te₇, CdSe.In₂Te₃, CdTe.Sn₂S₆, CdTe.Sn₂Te₆, CdTe.In₂Se₄, Au/PbS.Sn₂S₆, Au/PbSe.Sn₂S₆, Au/PbTe.Sn₂S₆, Au/CdS.Sn₂S₆, Au/CdSe.Sn₂S₆, Au/CdTe.Sn₂S₆, FePt/PbS.Sn₂S₆, FePt/PbSe.Sn₂S₆, FePt/PbTe.Sn₂S₆, FePt/CdS.Sn₂S₆, FePt/CdSe.Sn₂S₆, FePt/CdTe.Sn₂S₆, Au/PbS.SnS₄, Au/PbSe.SnS₄, Au/PbTe.SnS₄, Au/CdS.SnS₄, Au/CdSe.SnS₄, Au/CdTe.SnS₄, FePt/PbS.SnS₄ FePt/PbSe.SnS₄, Fe Pt/PbTe.SnS₄, FePt/CdS.SnS₄, FePt/CdSe.SnS₄, FePt/CdTe.SnS₄, Au/PbS.In₂Se₄ Au/PbSe.In₂Se₄, Au/PbTe.In₂Se₄, Au/CdS.In₂Se₄, Au/CdSe.In₂Se₄, Au/CdTe.In₂Se₄, FePt/PbS.In₂Se₄ FePt/PbSe.In₂Se₄, FePt/PbTe.In₂Se₄, FePt/CdS.In₂Se₄, FePt/CdSe.In₂Se₄, FePt/CdTe.In₂Se₄, CdSe/CdS.Sn₂S₆, CdSe/CdS.SnS₄, CdSe/ZnS.SnS₄,CdSe/CdS.Ge₂S₆, CdSe/CdS.In₂Se₄, CdSe/ZnS.In₂Se₄, Cu.In₂Se₄, Cu₂Se.Sn₂S₆, Pd.AsS₃, PbS.SnS₄, PbS.Sn₂S₆, PbS.Sn₂Se₆, PbS.In₂Se₄, PbS.Sn₂Te₆, PbS.AsS₃, ZnSe.Sn₂S₆, ZnSe.SnS₄, ZnS.Sn₂S₆, and ZnS.SnS₄ among others.

As used here the denotation ZnS.TiO₂ may refer to ZnS semiconductor nanocrystal 104 capped with TiO₂ inorganic capping agent. Charges on inorganic capping agent are omitted for clarity. This nomenclature [semiconductor nanocrystal].[inorganic capping agent] is used throughout this description. The specific percentages of semiconductor nanocrystal 104 precursors and inorganic capping agent may vary between different types of PCCN 102.

Structure of PCCN

FIG. 2 shows an embodiment of nanorod configuration 200 of PCCN 102 including first semiconductor nanocrystal 202 and second semiconductor nanocrystal 204 capped with first inorganic capping agent 206 and second inorganic capping agent 208, respectively. As an example, PCCN 102 in nanorod configuration 200 may include three ZnS region and four Cu regions as first semiconductor nanocrystal 202 and second semiconductor nanocrystal 204, respectively, where first semiconductor nanocrystal 202 may be larger than each of the four second semiconductor nanocrystal 204 of nanorod configuration 200. In other embodiments, the different regions with different materials may have the same lengths, and there can be any suitable number of different regions. The number of regions per nanorod superlattice in nanorod configuration 200 may vary according to the length of the nanorod. First inorganic capping agent 206 may include ReO₂, while W₂O₃ may be employed as second inorganic capping agent 208.

In addition, the shape of semiconductor nanocrystals 104 may improve photocatalytic activity of semiconductor nanocrystals 104. Changes in shape may expose different facets as reaction sites and may change the number and geometry of step edges where reactions may preferentially take place.

Other suitable configurations for PCCN 102 may be carbon nanotube, nanowire, nanospring, nanodendritic, spherical, tetrapod, core/shell and graphene configuration, among others.

Alignment Process for Forming Oriented Photoactive Material.

When a PCCN 102 interacts with an electromagnetic wave of frequency, i.e. when a PCCN 102 is being hit by photons, it can undergo a transition from an initial to a final state of energy difference through the coupling of the electromagnetic field to the transition dipole moment (TDM). The process of single photon absorption is characterized by the TDM. The TDM is a vector and has to do with the differences in electric charge distribution between an initial and final state of a PCCN 102. When this transition is from a lower energy state to a higher energy state, this results in the absorption of a photon. A transition from a higher energy state to a lower energy state, results in the emission of a photon.

The TDM may describe in which direction the electric charge within a PCCN 102 shifts during absorption of a photon. The amplitude of TDM is the transition moment between the initial (i) and final (f) states, and may be calculated as <f|V|i>, where “f” may be the wavefunction of the final state of PCCN 102, “i” may be the wavefunction of the initial state of PCCN 102, “V” may be the disturbance or transition dipole moment=mu*E (where “mu” may be the dipole moment of PCCN 102 in initial state, and “E” may be the electric part of the electromagnetic field). V is the electric dipole moment operator, a vector operator that is the sum of the position vectors of all charged particles weighted with their charge.

The TDM direction in the molecular framework defines the direction of transition polarization, and its square determines the strength of the transition.

FIG. 3 illustrates dipole moment characterization 300 within PCCN 102, according to an embodiment, describing the axis of the nanocrystal along which the electrons interact with the electromagnetic field of an incident photon. The TDM 302 relates the interaction of PCCN 102 to the polarization of incident light.

TDM 302 is a vector in the molecular framework, characterized both by its direction and its probability. The absorption probability for linearly polarized light is proportional to the cosine square of the angle between the electric vector of the electromagnetic wave and TDM 302; light absorption may be maximized if they are parallel, and no absorption may occur if they are perpendicular.

Therefore, by controlling the orientation of PCCN 102 employed in a light harvesting system, an increase in the efficiency of light absorption and hence, an increase in the energy conversion may be achieved. For this purpose oriented photoactive materials may be formed applying orientational forces to PCCN 102 during deposition and/or after they are deposited onto a suitable substrate.

Alignment Methods

In an embodiment, semiconductor nanocrystals 104 may be deposited and thermally treated on a suitable substrate, employing known in the art suitable methods (e.g. spraying deposition and annealing methods). For these methods, suitable substrates may include non-porous substrates and porous substrates, which may additionally be optically transparent in order to allow PCCN 102 to receive more light. Suitable non-porous substrates may include polydiallyldimethylammonium chloride (PDDA), polyethylene terephthalate (PET), and silicon, while suitable porous substrates may include TiO₂, glass frits, fiberglass cloth, porous alumina, and porous silicon. Suitable porous substrates may additionally exhibit a pore size sufficient for a gas to pass through at a constant flow rate. Suitable substrates may be planar or parabolic, individually controlled planar plates, or a grid work of plates.

According to an embodiment, semiconductor nanocrystals 104 may be applied to the substrate by means of a spraying device during a period of time depending on preferred thickness of semiconductor nanocrystal 104 composition applied on the substrate.

FIG. 4 is a flowchart of alignment method 400 for forming oriented photocatalyst semiconductor surfaces, according to an embodiment. Alignment method 400 for forming oriented photocatalyst semiconductor surfaces may include a deposition 402 of PCCN 102 on a suitable substrate, such as substrates mentioned in FIG. 3.

According to an embodiment, PCCN 102 may be deposited on the substrate by means of a spraying device during a period of time depending on preferred thickness of PCCN 102 composition deposited on the substrate. As a result of the spraying deposition, a photoactive material may be formed.

Other deposition 402 methods of PCCN 102 may include plating, chemical synthesis in solution, chemical vapor deposition (CVD), spin coating, plasma enhanced chemical vapor deposition (PECVD), laser ablation, thermal evaporation, molecular beam epitaxy, electron beam evaporation, pulsed laser deposition (PLD), sputtering, reactive sputtering, atomic layer deposition, sputter deposition, reverse Lang-muir-Blodgett technique, electrostatic deposition, spin coating, inkjet deposition, laser printing (matrices), and the like.

Subsequently, PCCN 102 within the photoactive material may be oriented by the application of orientational forces 404. Afterwards, PCCN 102 may pass through a thermal treatment 406 employing a convection heater, with temperatures less than between about 200 to about 350° C., to produce crystalline films from the PCCN 102. A thermal treatment 406 may yield, for example, ordered arrays of PCCN 102 within an inorganic matrix, hetero-alloys, or alloys.

FIG. 5 depicts alignment process 500 employing electric fields to orient the electric dipole moment (EDM 502) of PCCN 102, depicted by electric field lines 504, which might be an example of application of orientational forces 404.

Molecules including more than one type of atoms generally may have the tendency to form bonds where electrons are not shared equally. In this kind of molecules a region with high electron density and a region with low electron density may be found.

PCCN 102 may include atoms of different electronegativity, which makes them polar molecules, as such they may include a positively charged region, which may include a lower concentration of atoms with low electronegativity, and a negatively charged region, which may have a higher concentration of atoms with high electronegativity. Accordingly, electron density may be higher in the space surrounding negatively charged region and lower in the spacer surrounding positively charged region, while PCCN 102 molecules remain neutral as a whole. Negatively charged region may include a negatively charged center, about which the negative charge is centered. Similarly, positively charged region may include a positive charged center, about which the positive charge is centered. If the locations of negatively charged center and positive charged center are not coincident, PCCN 102 molecules include an EDM 502. The magnitude of EDM 502 may be equal to the distance between positive charged center and negatively charged center multiplied by the magnitude of the charge at either charge region (positively charged region or negatively charged region). The direction of EDM 502 may depend on the structure and composition of PCCN 102, generally pointing towards negatively charged region.

In an embodiment, the photoactive material 506, including PCCN 102, may be exposed to an external electric field. The EDM 502 of PCCN 102 may interact with the external electric field, causing PCCN 102 to rotate in such a way that the energy of EDM 502 in external electric field may be minimized. In many cases, this means that EDM 502 of PCCN 102 may be parallel to the electric field lines 504 and form an oriented photoactive material 508 which may be employed as an oriented photocatalyst semiconductor surface that may allow to predict the polarity of the light, for a more efficient interaction with the oriented photoactive material 508 and increase the light harvesting efficiency. The EDM 502 of the nanocrystals is along the same axis, the rods are oriented in the same angle on the substrate, all in the same orientation.

According to another embodiment, alignment process 500 may be controlled using charged ligands. By controlling the charged ligands of the PCCN 102, specific orientations of the PCCN 102 may also be obtained.

In another embodiment, methods for the application of orientational forces 404 may include known in the art combing deposition technique, which may include a slowly wicking away solvent of the solution including the semiconductor nanocrystals 104 to be deposited, so that at the meniscus interface, semiconductor nanocrystals 104 experience a directional force along the direction of the wicking action.

In another embodiment, photoactive material 506 may pass through a surface charge. Some of the faces of PCCN 102 may be ionic in nature and by having a charged substrate it may be possible to predefine which face or faces of PCCN 102 interact or are attached to the substrate during deposition. Cationic faces may be attracted to negatively charged substrates and anionic faces may be attracted towards positively charged substrates. For example, in PCCN 102 including Cd²⁺ or Zn²⁺, are generally cationic in nature and a negatively-charged substrate may preferentially attract these crystal faces, resulting in some degree of orientation of PCCN 102.

In yet another embodiment, photoactive material 506 may be oriented employing a Langmuir Blodgett film, which may be formed by employing Langmuir Blodgett method, resulting in the alignment of a thin film monolayer of PCCN 102 along 2 axes (1D or 2D orientation) to form oriented photoactive material 508.

Employing the Langmuir Blodgett method a PCCN 102 monolayer may be formed on a water surface by compression and subsequently the PCCN 102 monolayer may be transferred onto a suitable substrate by a controlled removal of the water sub-phase.

In an embodiment, photoactive material 506 may be oriented by controlling the surface-ligands. By controlling the ligands on the surface of the PCCN 102 and ligands on the surface of the substrate, specific orientations of the PCCN 102 to the substrate may be obtained.

PCCN 102 may include one or more alignment ligands associated with the PCCN 102. The structurally ordering of the plurality of PCCN 102 may be achieved by the interaction of a first alignment ligand on a first PCCN 102 with a second alignment ligand on an adjacent PCCN 102. Generally the first and second alignment ligands may be complementary binding pairs. Optionally, both complements of the binding pair are provided on the same molecule (e.g., a multifunctional molecule). In some embodiments, a single chemical entity can be used as the first and second alignment ligands. Alternatively, the two halves of the complementary binding pair can be provided on different compositions, such that the first and second alignment ligands are differing molecules.

Interacting the first and second alignment ligands to achieve the selective orientation of the plurality of PCCN 102, can be performed, for example, by heating and cooling the plurality of PCCN 102. In embodiments in which the first and second alignment ligands further include a crosslinking or polymerizable element, interacting the alignment ligands may include the step of crosslinking or polymerizing the first and second alignment ligands, e.g., to form a matrix.

As a further embodiment of the methods of the present disclosure, the plurality of oriented PCCN 102 may be affixed to a substrate or surface. Optionally, the first and second alignment ligands may be removed after affixing the aligned PCCN 102, to produce a plurality of oriented PCCN 102 on a substrate.

After alignment process 500, oriented photoactive material 508 may be cut into films to be used as oriented photocatalyst semiconductor surfaces in energy conversion applications, including photocatalytic water splitting and carbon dioxide reduction.

FIG. 6 depicts an embodiment of oriented PCCN 600 in nanorod configuration 200 showing oriented TDM 302 receiving light 602. TDM 302 of oriented PCCN 600 may be oriented at a fi angle 604 from an axis 606 normal to the upper surface of substrate 608 onto which PCCN 102 has been deposited. Additionally, in order for light 602 to be absorbed by PCCN 102, light 602 may have a non-zero component of its electric field vector in line with TDM 302 of PCCN 102.

Oriented Photoactive Material

FIG. 7 illustrates an embodiment of oriented photoactive material 508, including oriented PCCN 600 in nanorod configuration 200 upon substrate 608. Oriented PCCN 600 in oriented photoactive material 508 may also exhibit carbon nanotube, nanosprings and nanowire configuration, among others.

In order to measure the performance of oriented photoactive material 508, devices such as transmission electron microscopy (TEM), and energy dispersive X-ray (EDX), among others, may be utilized. Performance of oriented photoactive material 508 may be related to light absorbance, charge carriers mobility and energy conversion efficiency. Performance of oriented photoactive material 508 may be related to light absorbance, charge carriers mobility and energy conversion efficiency.

Oriented photoactive material 508 may be employed in any of a number of devices and applications, including, but not limited to, various photovoltaic devices, optoelectronic devices (LEDs, lasers, optical amplifiers), light collectors, photodetectors and/or the like. Oriented photoactive material 508 may be also employed in energy conversion processes, such as water splitting and carbon dioxide reduction, among others

System Configuration and Functioning

FIG. 8 shows charge separation process 800 that may occur during water splitting process and carbon dioxide reduction.

The energy difference between valence band 802 and conduction band 804 of a semiconductor nanocrystal 104 is known as band gap 806. Valence band 802 refers to the outermost electron 808 shell of atoms in semiconductor nanocrystals 104 and insulators in which electrons 808 are too tightly bound to the atom to carry electric current, while conduction band 804 refers to the band of orbitals that are high in energy and are generally empty. Band gap 806 of semiconductor nanocrystals 104 should be large enough to drive water splitting process reactions, but small enough to absorb a large fraction of light 602 wavelengths. The manifestation of band gap 806 in optical absorption is that only photons with energy larger than or equal to band gap 806 are absorbed.

When light 602 with energy equal to or greater than that of band gap 806 makes contact with semiconductor nanocrystals 104 in oriented photoactive material 508, electrons 808 are excited from valence band 802 to conduction band 804, leaving holes 810 behind in valence band 802, a process triggered by photo-excitation 812. Changing the materials and shapes of semiconductor nanocrystals 104 may enable the tuning of band gap 806 and band-offsets to expand the range of wavelengths usable by semiconductor nanocrystal 104 and to tune the band positions for redox processes.

Water Splitting Process:

For water splitting process, the photo-excited electron 808 in semiconductor nanocrystal 104 should have a reduction potential greater than or equal to that necessary to drive the following reaction:

2H₃O⁺+2e⁻→H₂+2H₂O (1)

The above stated reaction may have a standard reduction potential of 0.0 eV vs. Standard Hydrogen Electrode (SHE), or standard hydrogen potential of 0.0 eV. Hydrogen (H₂) molecule in water may be reduced when receiving two photo-excited electrons 808 moving from valence band 802 to conduction band 804. On the other hand, the photo-excited hole 810 should have an oxidation potential greater than or equal to that necessary to drive the following reaction:

6H₂O+4h⁺→O₂+4H₃O⁺ (2)

The above stated reaction may exhibit a standard oxidation potential of −1.23 eV vs. SHE. Oxygen (O₂) molecule in water may be oxidized by four holes 810. Therefore, the absolute minimum band gap 806 for semiconductor nanocrystal 104 in a water splitting reaction is 1.23 eV. Given over potentials and loss of energy for transferring the charges to donor and acceptor states, the minimum energy may be closer to 2.1 eV. The wavelength of the irradiation light may be required to be about 1010 nm or less, in order to allow electrons 808 to be excited and jump over band gap 806.

Electrons 808 may acquire energy corresponding to the wavelength of the absorbed light. Upon being excited, electrons 808 may relax to the bottom of conduction band 804, which may lead to recombination with holes 810 and therefore to an inefficient water splitting process. For efficient charge separation process 800, a reaction has to take place to quickly sequester and hold electron 808 and hole 810 for use in subsequent redox reactions used for water splitting process.

In one embodiment, semiconductor nanocrystal 104 in oriented photoactive material 508 may be capped with first inorganic capping agent 206 and second inorganic capping agent 208 as a reduction photocatalyst and an oxidative photocatalyst, respectively. Following photo-excitation 812 to conduction band 804, electron 808 can quickly move to the acceptor state of first inorganic capping agent 206 and hole 810 can move to the donor state of second inorganic capping agent 208, preventing recombination of electrons 808 and holes 810. First inorganic capping agent 206 acceptor state and second inorganic capping agent 208 donor state lie energetically between the band edge states and the redox potentials of the hydrogen and oxygen producing half-reactions. The sequestration of the charges into these states may also physically separate electrons 808 and holes 810, in addition to the physical charge carriers' separation that occurs in the boundaries between individual semiconductor nanocrystals 104. Being more stable to recombination in the donor and acceptor states, charge carriers may be efficiently stored for use in redox reactions required for photocatalytic water splitting process.

According to an embodiment, for water splitting process, a reaction vessel may be used. The reaction vessel may include oriented photoactive material 508 submerged in water. Light 602 coming from a light source, which may be the sun, may enter to the reaction vessel through a window. Subsequently, light 602 may come in contact with oriented photoactive material 508 and may produce charge separation process 800 (explained in FIG. 8) and charge transfer in the boundary between oriented photoactive material 508 and water; consequently, splitting water into hydrogen gas and oxygen gas.

The water splitting process may be characterized by the efficiency of converting light 602 energy into chemical energy. Hydrogen gas, when reacted with oxygen gas liberates 2.96 eV per water molecule. Thus, the amount of chemical energy can be determined by multiplying the number of hydrogen molecules generated by 2.96 eV. The energy of solar light 602 is defined as the amount of energy in light 602 having a wavelength from about 300 nm to about 800 nm. A typical solar intensity as measured at the Earth's surface, thus defined, is about 500 watts/m². The efficiency of water splitting process can be calculated as:

Efficiency=[(2.96 eV×(1.602×10⁻¹⁹J/eV)−N/t](I_{L}×A_{L}) (3)

where t is the time in seconds, I_{L} is the intensity of light 602 (between 300 nm and 800 nm) in watts/m², A_{L} is the area of light 602 entering reaction vessel in m², N is the number of hydrogen molecules generated in time t, and 1 watt=1 J/s.

In one embodiment, the water splitting process may take place in the boundary between oriented photoactive material 508 and water, oriented photoactive material 508 may include PCCN 102 in nanorod configuration 200. PCCN 102 may include semiconductor nanocrystal 104 capped with first inorganic capping agent 206 and second inorganic capping agent 208, acting as a reduction photocatalyst and oxidation photocatalyst respectively. When light 602 emitted by a light source makes contact with semiconductor nanocrystals 104, charge separation process 800 and charge transfer process may take place between semiconductor nanocrystal 104, first inorganic capping agent 206, second inorganic capping agent 208 and water. As a result, hydrogen may be reduced by electrons 808 moving from valence band 802 to conduction band 804 when electrons 808 may be transferred via first inorganic capping agent 206 to water, producing hydrogen gas molecules. On the other hand, oxygen may be oxidized by holes 810, when holes 810 are transferred via second inorganic capping agent 208 to water, resulting in the production of oxygen gas molecules.

Carbon Dioxide Reduction Process:

For carbon dioxide reduction process, band gap 806 of semiconductor nanocrystals 104 should be large enough to drive carbon dioxide reduction reactions but small enough to absorb a large fraction of light wavelengths. Band gap 806 of PCCN 102 employed in the reduction of carbon dioxide should be at least 1.33 eV, which corresponds to absorption of solar photons of wavelengths below 930 nm. Considering the energy loss associated with entropy change (87 J/mol·K) and other losses involved in carbon dioxide reduction (forming methane and water vapor), band gap 806 between about 2 and about 2.4 eV may be preferred. The manifestation of band gap 806 in optical absorption is that only photons with energy larger than or equal to band gap 806 are absorbed.

Electrons 808 may acquire energy corresponding to the wavelength of absorbed light 602. Upon being excited, electrons 808 may relax to the bottom of conduction band 804, which may lead to recombination with holes 810 and, therefore, to an inefficient charge separation process 800.

According to one embodiment, to achieve an charge separation process 800 for a carbon dioxide reduction process, semiconductor nanocrystal 104 in oriented photoactive material 508 may be capped with first inorganic capping agent 206 and second inorganic capping agent 208 as a reduction photocatalyst and an oxidative photocatalyst, respectively. Following photo-excitation 812 to conduction band 804, electron 808 can quickly move to the acceptor state of first inorganic capping agent 206 and hole 810 can move to the donor state of second inorganic capping agent 208, preventing recombination of electrons 808 and holes 810. First inorganic capping agent 206 acceptor state and second inorganic capping agent 208 donor state lie energetically between the limits of band gap 806 and the redox potentials of the hydrogen oxidation and carbon dioxide reduction reactions. By being more stable to recombination in the donor and acceptor states, charge carriers may be stored for use in redox reactions required for a more efficient charge separation process 800, and hence, a more productive carbon dioxide reduction process.

When semiconductor nanocrystals 104 in oriented photoactive material 508 are irradiated with photons having a level of energy greater than band gap 806 of oriented photoactive material 508, electrons 808 may be excited from valence band 802 into conduction band 804, leaving holes 810 behind in valence band 802. Excited electrons 808 may reduce carbon dioxide molecules into methane, while holes 810 may oxidize hydrogen gas molecules. Oxidized hydrogen molecules may react with carbon dioxide and form water and methane via a series of reactions that may be summarized by the equations on table 1:

TABLE 1
Carbon dioxide reduction equations

| Equation | Product |
|---|---|
| O₂ + 2H⁺ + 2e⁻ → HCOOH | Formic acid |
| COOH + 2H⁺ + 2e⁻ → HCHO + H₂O | Formaldehyde |
| HCHO + 2H⁺ + 2e⁻ → CH₃OH⁻ | Methanol |
| CH₃OH + 2H⁺ + 2e⁻ → CH₄ + H₂O | Methane |

According to table 1, in the carbon dioxide reduction process, carbon dioxide, in the presence of hydrogen, may be photo-catalytically reduced into methane and water. Electrons 808 may be obtained from oriented photoactive material 508 and hydrogen atoms may be obtained from hydrogen gas. Beginning from adsorbed carbon dioxide, formic acid (HCOOH) may be formed by accepting two electrons 808 and adding two hydrogen atoms. Then, formaldehyde (HCHO) and water molecules may be formed from the reduction of formic acid by accepting two electrons 808 and adding two hydrogen atoms. Subsequently, methanol (CH₃OH) may be formed when formaldehyde accepts two electrons 808 and two hydrogen atoms may be added to formaldehyde. Finally, methane may be formed when methanol accepts two electrons 808 and two hydrogen atoms are added to methanol. In addition, water may be formed as a byproduct of the reaction.

The reduction of carbon dioxide to methane requires reducing the chemical state of carbon from C (4+) to C (4−). Eight electrons 808 are required for the production of each methane. Taken as a whole, eight hydrogen atoms and eight electrons 808 progressively transfer to one adsorbed carbon dioxide molecule resulting in the production of one methane molecule. Similarly, oxygen released from carbon dioxide may react with free hydrogen radicals and form water vapor molecules.

According to an embodiment, for carbon reduction process, a reaction vessel may be used. The reaction vessel may include oriented photoactive material 508. Carbon dioxide may be introduced into the reaction vessel via an inlet line. Similarly, hydrogen gas may be injected into the reaction vessel by another inlet line.

Light 602 coming from a light source, which may be the sun, may enter to the reaction vessel through a window. Carbon dioxide and hydrogen gas may pass through oriented photoactive material 508 prior to entering into the reaction vessel. Light 602 may react with oriented photoactive material 508 and may produce charge separation process 800 (explained in FIG. 8) in the boundary of oriented photoactive material 508. Carbon dioxide may be reduced and hydrogen gas may be oxidized by a series of reactions until methane and water vapor are produced.

Light Polarization System:

Any suitable light source may be employed to provide light 602 for generating water splitting process to produce hydrogen and oxygen. A preferable light source is sunlight, including infrared light which may be used to heat water and also ultraviolet and visible light which may be used in water splitting process. The ultraviolet light and visible light may also heat water, directly or indirectly. Light 602 may be diffuse, direct, or both, filtered or unfiltered, modulated or unmodulated, attenuated or unattenuated. Preferably, light 602 may be polarized to increase the intensity and achieve a specific orientation towards oriented photoactive material 508. The polarizing system may include any suitable combination of mirrors, or any other suitable reflective surface, to increase the intensity of light 602. The increase in the intensity of light 602 may be characterized by the intensity of light 602 having from about 300 to about 1500 nm (e.g., from about 300 nm to about 800 nm) in wavelength. The polarizing system may increase the intensity of light 602 by any factor, preferably by a factor greater than about 2 to about 25.

Employing the polarization system, a partial linear polarization of light 602 may be achieved after reflecting off a single mirror face, so at least one mirrored surface may be necessary to achieve polarization. This is the preferred method for achieving linearly-polarized light. However, in some embodiments, more than one mirrored face may be helpful to best guide the incident light to focus on oriented photoactive material 508. To achieve linearly-polarized light, the first, polarizing mirror may be kept at Brewster's angle relative to the direction of the sun. In some embodiments, the mirror may have a thin glass layer on top, which may serve as a protective layer to the reflective metal surface. For most applications the protective glass layer may be thin enough, to avoid undesired optical interference. Furthermore, in some embodiments, the system may optionally include a sun-tracking system that allows the mirror collecting incident light to be always at Brewster's angle relative to the sun. The addition of the sun tracking system may allow the optimal recollection of light at all times.

FIG. 9 shows light polarization method 900. In light polarization method 900, randomly polarized incident light 902 irradiated by light source 904, which may be the sun, may become linearly polarized light 906, if randomly polarized incident light 902 hits the surface of mirror 908 at a fi angle 604, which is equivalent to the Brewster's angle of incidence of mirror 908. Oriented photoactive material 508 may be positioned in such a way that alpha angle 910, at which linearly polarized light 906 reaches oriented photoactive material 508, allows the optimal absorption of linearly polarized light 906. A sun tracking system may be used to keep fi angle 604 and alpha angle 910 in a suitable range, such that efficiency may be increased at all times.

FIG. 10 shows multiple mirror configuration 1000, which may be an embodiment of light polarization method 900. In multiple mirror configuration 1000, randomly polarized incident light 902 may be collected by tracking mirror 1002, which tracks the movement of light source 904 to collect and polarize sunlight, maintaining fi angle 604 equal to Brewster's angle of incidence. Then, first steering mirror 1004 and second steering mirror 1006 may direct linearly polarized light 906 towards oriented photoactive material 508 at the optimum alpha angle 910 of incidence. First steering mirror 1004 and second steering mirror 1006 may be capable of changing their relative position in order to ensure that at all times alpha angle 910 is maintained at optimal or preferred values. By the addition of first steering mirror 1004 and second steering mirror 1006, oriented photoactive material 508 may remain in a fixed position.

FIG. 11 shows focusing mirrors configuration 1100, which may be an embodiment of light polarization method 900. In an embodiment, randomly polarized incident light 902 may be collected by tracking mirror 1002, which tracks the movement of light source 904 to collect and polarize sunlight, maintaining fi angle 604 equal to Brewster's angle of incidence. Then first focusing steering mirror 1102 and second focusing steering mirror 1104 may direct focused linearly polarized light 1106 towards oriented photoactive material 508. By focusing linearly polarized light 906 it may be possible to increase the efficiency and lower the necessary active surface of oriented photoactive material 508.

The systems explained above may be employed to polarize sunlight to collect solar energy and orient the light rays for maximum absorption and energy conversion on oriented photocatalytic surfaces.

FIG. 12 represents photosynthetic system 1200 to perform water splitting process and carbon dioxide reduction process, employing oriented photoactive material 508. Photosynthetic system 1200 may include reaction vessel A 1202, gas collecting chamber 1220 and reaction vessel B 1206.

In photosynthetic system 1200, reaction vessel A 1202 includes oriented photoactive material 508 that may be submerged in water 1208. Randomly polarized incident light 902 coming from light source 904 may be polarized by a light polarizing system in focusing mirrors configuration 1100 (explained in FIG. 11). The light polarizing system in focusing mirrors configuration 1100 may reflect randomly polarized incident light 902 and may direct focused linearly polarized light 1106 at reaction vessel A 1202 through a window. Subsequently, focused linearly polarized light 1106 may come in contact with oriented photoactive material 508 and may produce charge separation process 800 for splitting water into hydrogen gas 1216 and oxygen gas 1218. In one embodiment, solar reflector 1210 may be positioned at any side of reaction vessel A 1202 to reflect focused linearly polarized light 1106 back to reaction vessel A 1202 and re-utilize focused linearly polarized light 1106.

A continuous flow of water 1208 may enter reaction vessel A 1202 through inlet line 1212 to a region including oriented photoactive material 508. Preferably, a heater 1214 may be connected to reaction vessel A 1202 in order to produce heat, so that water 1208 may boil, facilitating the migration of hydrogen gas 1216 and oxygen gas 1218 from reaction vessel A 1202 to gas collecting chamber 1220 through opening 1222. Heater 1214 may be set to a temperature of at least 100° C. Heater 1214 may be powered by different energy supplying devices. Preferably, heater 1214 may be powered by renewable energy supplying devices, such as photovoltaic cells, or by energy stored employing the system and method from the present disclosure. Materials for the walls of reaction vessel A 1202 may be selected based on the reaction temperature.

After reaction vessel A 1202, hydrogen gas 1216 and oxygen gas 1218 may migrate through opening 1222 to gas collecting chamber 1220. Gas collecting chamber 1220 may include hydrogen permeable membrane 1224 (e.g. silica membrane) and oxygen permeable membrane 1226 (e.g. silanized alumina membrane). Oxygen permeable membrane 1226 may absorb only oxygen gas 1218 and subsequently transfer oxygen gas 1218 into oxygen storage tank 1228 or into any other suitable storage equipment. Hydrogen permeable membrane 1224 may absorb hydrogen gas 1216 and subsequently transfer hydrogen gas 1216 into reaction vessel B 1206 through oriented photoactive material 508. Flow of hydrogen gas 1216, oxygen gas 1218 and water 1208 may be controlled by one or more valves, pumps or other flow regulators.

Photosynthetic system 1200 may operate in conjunction with a combustion system that produces carbon dioxide 1230 as a byproduct. In an embodiment, photosynthetic system 1200 may be employed to take advantage of carbon dioxide 1230 produced by one or more boilers 1232 during a manufacturing process. Boiler 1232 may be connected to reaction vessel B 1206 by inlet line B 1234 that may allow a continuous flow of carbon dioxide 1230 gas through oriented photoactive material 508 along with hydrogen gas 1216 into reaction vessel B 1206.

Randomly polarized incident light 902 coming from light source 904 may be polarized by a light polarizing system in focusing mirrors configuration 1100 (explained in FIG. 11). The light polarizing system in focusing mirrors configuration 1100 may reflect randomly polarized incident light 902 and may direct focused linearly polarized light 1106 at reaction vessel B 1206 through a window. Carbon dioxide 1230 and hydrogen gas 1216 may pass through oriented photoactive material 508 prior to entering into reaction vessel B 1206. Focused linearly polarized light 1106 may react with oriented photoactive material 508 to produce charge separation process 800. In an embodiment, solar reflector 1210 may be positioned at any side of reaction vessel B 1206 to reflect focused linearly polarized light 1106 back to reaction vessel B 1206 and re-use focused linearly polarized light 1106.

When carbon dioxide 1230 and hydrogen gas 1216 come in contact with oriented photoactive material 508, carbon dioxide reduction process may take place through reactions summarized in table 1 (explained in FIG. 8). Optionally, a heater (not shown in FIG. 12) may be employed to increase the temperature in reaction vessel B 1206.

After carbon dioxide reduction process, the produced methane 1236 may exit reaction vessel B 1206 through methane permeable membrane 1238 (e.g. polyimide resin membrane) to be subsequently stored in methane storage tank 1240 or any suitable storage medium or may be directly used as fuel by boiler 1232, according to the manufacturing process needs of the industry that applies photosynthetic system 1200.

Water vapor 1242 may exit reaction vessel B 1206 through water vapor permeable membrane 1244 (e.g. polydimethylsiloxane membrane) and may be transferred to water condenser 1246 where liquid water 1208 may be obtained. Valves, pumps and/or monitoring devices may be added in order to measure and regulate pressure and/or flow rate. Flow rate of carbon dioxide 1230 and hydrogen gas 1216 into reaction vessel B 1206 may be adjusted depending on reaction time between carbon dioxide 1230, hydrogen gas 1216 and oriented photoactive material 508 needed. Optionally, a gas sensor device (not shown in this figure) may be installed near reaction vessel B 1206 to identify any methane 1236 leakage.

Liquid water may be employed for different purposes in the manufacturing process. In an embodiment, liquid water may be recirculated through pipeline 1248 to supply water to reaction vessel A 1202. Stored methane 1236 produced in photosynthetic system 1200 may be burned as industrial fuel for boilers 1232 and kilns, residential fuel, vehicle fuel, and/or as fuel for turbines for electricity production.

According to various embodiments, one or more walls of reaction vessel A 1202 and reaction vessel B 1206 may be formed of glass or other transparent material, so that focused linearly polarized light 1106 may enter reaction vessel A 1202 and reaction vessel B 1206. It may also be possible that most or all of the walls of reaction vessel A 1202 and reaction vessel B 1206 are transparent such that focused linearly polarized light 1106 may enter from many directions. In another embodiment, reaction vessel A 1202 and reaction vessel B 1206 may have one or more transparent sides to allow the incident radiation to enter and the other sides may have a reflective interior surface which reflects the majority of the solar radiation.

While various aspects and embodiments have been disclosed, other aspects and embodiments are contemplated. The various aspects and embodiments disclosed are for purposes of illustration and are not intended to be limiting, with the true scope and spirit being indicated by the following claims.

### EXAMPLES

Example #1 is an embodiment of photosynthetic system 1200 where gas collecting chamber 1220 is not included, in which oxygen gas 1218 and hydrogen gas 1216 from reaction vessel A 1202 may be transferred directly into reaction vessel B 1206. Hydrogen gas 1216 may pass through hydrogen permeable membrane 1224 in order to be transferred into reaction vessel B 1206; oxygen gas 1218 may pass through oxygen permeable membrane 1226 in order to be collected into an oxygen storage tank 1228.

It should be understood that the present disclosure is not limited in its application to the details of construction and arrangements of the components set forth here. The present disclosure is capable of other embodiments and of being practiced or carried out in various ways. Variations and modifications of the foregoing are within the scope of the present disclosure. It also being understood that the invention disclosed and defined here extends to all alternative combinations of two or more of the individual features mentioned or evident from the text and/or drawings. All of these different combinations constitute various alternative aspects of the present invention. The embodiments described here explain the best modes known for practicing the invention and will enable others skilled in the art to utilize the invention.

Thus the scope of the invention should be determined by the appended claims and their legal equivalents, and not by the examples given.

While various aspects and embodiments have been disclosed, other aspects and embodiments are contemplated. The various aspects and embodiments disclosed are for purposes of illustration and are not intended to be limiting, with the true scope and spirit being indicated by the following claims.

The embodiments described above are intended to be exemplary. One skilled in the art recognizes that numerous alternative components and embodiments that may be substituted for the particular examples described herein and still fall within the scope of the invention.

What's claimed is:

1. A method for water splitting and carbon dioxide reduction comprising:
forming photocatalytic capped colloidal nanocrystals, wherein each photocatalytic capped colloidal nanocrystal includes a first semiconductor nanocrystal capped with a first inorganic capping agent;
depositing the formed photocatalytic capped colloidal nanocrystals onto a first substrate and a second substrate, thereby creating first and second photoactive materials;
orienting the photocatalytic capped colloidal nanocrystals of the first photoactive material;
orienting the photocatalytic capped colloidal nanocrystals of the second photoactive material;
absorbing irradiated light with an energy equal to or greater than the band gap of the semiconductor nanocrystals by the first photoactive material to create charge carriers in a conduction band and holes in a valence band of the photocatalytic capped colloidal nanocrystals of the first photoactive material;
passing water through a first reaction vessel so that the water reacts with the first photoactive material to form hydrogen and oxygen, wherein the charge carriers in the conduction band reduce hydrogen molecules from the water and the holes in the valence band oxidize oxygen molecules from the water;
separating the hydrogen from the oxygen using a hydrogen permeable membrane and an oxygen permeable membrane;
passing the separated hydrogen from the first reaction vessel into a second reaction vessel;
passing carbon dioxide into the second reaction vessel;
absorbing irradiated light with an energy equal to or greater than the band gap of the semiconductor nanocrystals by the second photoactive material to create charge carriers in a conduction band and holes in a valence band of the photocatalytic capped colloidal nanocrystals of the second photoactive material;
reacting the carbon dioxide and the hydrogen with the second photoactive material in the second reaction vessel so that the charge carriers in the conduction band reduce carbon dioxide into methane and the holes in the valence band oxidize the hydrogen into water vapor; and
collecting the methane using a methane permeable membrane.

2. The method of claim 1, further comprising:
collecting the water vapor using a water vapor permeable membrane;
transferring the collected water vapor to a condenser through an outlet line connected to the second reaction vessel to obtain liquid water; and
transferring the liquid water to the first reaction vessel.

3. The method of claim 1, wherein the carbon dioxide is produced by a combustion system that is connected to the second reaction vessel.

4. The method of claim 3, further comprising:
transferring the methane to the combustion system so that the methane may be used as fuel in the combustion system.

5. The method of claim 1, further comprising:
polarizing the irradiated light with at least one first mirror before the first photoactive material absorbs the irradiated light; and
polarizing the irradiated light with at least one second mirror before the second photoactive material absorbs the irradiated light.

6. The method of claim 5, further comprising:
steering the at least one first mirror so that the at least one first mirror maintains Brewster's angle relative to the sun; and
steering the at least one second mirror so that the at least one second mirror maintains Brewster's angle relative to the sun.

7. The method of claim 6, wherein the at least one first mirror and the at least one second mirror are steered using a sun tracking system.

8. The method of claim 5, wherein the at least one first mirror and the at least one second mirror are focusing mirrors.

9. The method of claim 6, further comprising:
steering a third mirror so that the polarized light from the at least one first mirror is directed at the first photoactive material at an angle that facilitates absorption; and
steering a fourth mirror so that the polarized light from the at least one second mirror is directed at the second photoactive material at an angle that facilitates absorption.

10. The method of claim 1, wherein forming photocatalytic capped colloidal nanocrystals comprises:
growing semiconductor nanocrystals by employing a template-driven seeded growth method; and
capping the semiconductor nanocrystals with an inorganic capping agent in a polar solvent to form photocatalytic capped colloidal nanocrystals.

11. The method of claim 10, wherein growing semiconductor nanocrystals by employing the template-driven seeded growth method comprises:
depositing a seed crystal on a substrate; and
growing the semiconductor nanocrystal from the seed crystal using molecular beam epitaxy or chemical beam epitaxy so that the semiconductor nanocrystal grows according to the seed crystal's structure.

12. The method of claim 11, wherein capping the semiconductor nanocrystals with an inorganic capping agent in the polar solvent to form the photocatalytic capped colloidal nanocrystals comprises:
reacting semiconductor nanocrystals precursors in the presence of an organic capping agent to form organic capped semiconductor nanocrystals;
reacting the organic capped semiconductor nanocrystals with an inorganic capping agent;
adding immiscible solvents causing the dissolution of the organic capping agents and the inorganic capping agents so that organic caps on the semiconductor nanocrystals are replaced by inorganic caps to form inorganic capped semiconductor nanocrystals; and
performing an isolation procedure to purify the inorganic capped semiconductor nanocrystals and remove the organic capping agent.

13. The method of claim 1, wherein orienting the photocatalytic capped colloidal nanocrystals is performed by applying an electric field, and the direction of the electric field is substantially parallel with an electric dipole moment of the photocatalytic capped colloidal nanocrystals.

14. The method of claim 1, further comprising:
heating the water entering the first reaction vessel so that the water boils and is in a gaseous state when reacting with the first photoactive material in the first reaction vessel.

15. The method of claim 1, further comprising:
filtering unreacted water, the hydrogen, and the oxygen leaving the first reaction vessel.

16. The method of claim 1, wherein a shapes of the photocatalytic capped colloidal nanocrystals for the first and second photoactive materials are chosen based on a desired wavelength of the irradiated light usable by the semiconductor nanocrystals.

17. The method of claim 1, further comprising heating the second reaction vessel with a heater.

18. The method of claim 1, wherein each photocatalytic capped colloidal nanocrystals includes a second semiconductor nanocrystal capped with a second inorganic capping agent, the first inorganic capping agent acts as a reduction photocatalyst, and the second inorganic capping agent acts as an oxidation photocatalyst.

19. A method for water splitting and carbon dioxide reduction comprising:
absorbing irradiated light with an energy equal to or greater than the band gap of semiconductor nanocrystals in a first photoactive material to create charge carriers in a conduction band and holes in a valence band of photocatalytic capped colloidal nanocrystals of the first photoactive material;
passing water through a first reaction vessel so that the water reacts with the first photoactive material to form hydrogen and oxygen, wherein the charge carriers in the conduction band reduce hydrogen molecules from the water and the holes in the valence band oxidize oxygen molecules from the water;
separating the hydrogen from the oxygen using a hydrogen permeable membrane and an oxygen permeable membrane;
collecting the separated oxygen in an oxygen storage tank;
passing the separated hydrogen from the first reaction vessel into a second reaction vessel;
transferring carbon dioxide into the second reaction vessel from boiler that produces carbon dioxide through a combustion reaction;
absorbing irradiated light with an energy equal to or greater than the band gap of semiconductor nanocrystals in a second photoactive material to create charge carriers in a conduction band and holes in a valence band of photocatalytic capped colloidal nanocrystals of the second photoactive material;
reacting the carbon dioxide and the hydrogen with the second photoactive material in the second reaction vessel so that the charge carriers in the conduction band reduce carbon dioxide into methane and the holes in the valence band oxidize the hydrogen into water vapor;
separating the methane using a methane permeable membrane;
collecting the separated methane in a storage tank; and
recycling the water vapor to the first reaction vessel.

20. A photosynthetic system comprising:
first and second oriented photoactive materials, wherein the first and second oriented photoactive materials include oriented photocatalytic capped colloidal nanocrystals;
a first reaction vessel housing the first oriented photoactive material and configured to receive water through an inlet and facilitate a water splitting reaction that produces hydrogen and oxygen when the water reacts with the photocatalytic capped colloidal nanocrystals, wherein the water splitting reaction occurs when the photocatalytic capped colloidal nanocrystals absorb irradiated light to separate charge carriers of the first oriented photoactive material; and
a second reaction vessel housing the second oriented photoactive material and configured to receive carbon dioxide through a first inlet, receive hydrogen from the first reaction vessel, and facilitate a carbon dioxide reduction reaction and a hydrogen oxidization reaction that produces methane and water vapor, wherein the reaction begins when the photocatalytic capped colloidal nanocrystals of the second photoactive material absorb polarized light to separate charge carriers of the second oriented photoactive material.

21. The photosynthetic system of claim 20, further comprising:
a hydrogen-permeable membrane configured to separate the hydrogen from the oxygen in the first reaction vessel, wherein the hydrogen passes through the hydrogen-permeable membrane into the second reaction vessel.

22. The photosynthetic system of claim 21, further comprising:
a oxygen-permeable membrane configured to separate the oxygen from the hydrogen in the first reaction vessel, wherein the oxygen passes through the oxygen-permeable membrane into an oxygen storage tank.

23. The photosynthetic system of claim 22, wherein the hydrogen-permeable membrane and the oxygen-permeable membrane are included in a gas collecting chamber.

25. The photosynthetic system of claim 20, further comprising:
a methane-permeable membrane configured to separate the methane from the water vapor in the second reaction vessel, wherein the methane passes through the methane-permeable membrane into an methane storage tank.

25. The photosynthetic system of claim 20, further comprising:
a water condenser connected to the second reaction vessel and configured to convert water vapor into liquid water.

26. The photosynthetic system of claim 25, further comprising:
a water vapor permeable membrane configured to separate the water vapor from the methane in the second reaction vessel, wherein the water vapor passes through the water vapor permeable membrane the water condenser.

27. The photosynthetic system of claim 25, wherein the liquid water from the water condenser is transferred to the first reaction vessel.

28. The photosynthetic system of claim 20, further comprising
a first mirror that collects and linearly polarizes the irradiated light irradiated by the sun; and
a second mirror that collects and linearly polarizes the irradiated light irradiated by the sun

29. The photosynthetic system of claim 28, further comprising:
a first steering mirror that directs the linearly polarized light received from the first mirror toward the first oriented photoactive material at a first optimum angle of incidence, wherein the first optimum angle of incidence depends on the orientation of the photocatalytic capped colloidal nanocrystals of the first oriented photoactive material; and
a second steering mirror that directs the linearly polarized light received from the second mirror toward the second oriented photoactive material at a second optimum angle of incidence, wherein the second optimum angle of incidence depends on the orientation of the photocatalytic capped colloidal nanocrystals of the second oriented photoactive material.

26. The photosynthetic system of claim 28, wherein the first and second mirrors are connected to a sun tracking system so that the first and second mirrors receive sunlight at Brewster's angle.

27. The photosynthetic system of claim 28, wherein the first and second mirrors are focusing mirrors.

28. The photosynthetic system of claim 20, further comprising:
a first heater that heats the water entering the first reaction vessel; and
a second heater that heats the second reaction vessel.

29. The photosynthetic system of claim 20, further comprising:
a boiler that produces carbon dioxide through a combustion reaction, wherein the carbon dioxide produced by the boiler is transferred to the second reaction vessel.

30. The photosynthetic system of claim 29, wherein the methane produced in the second reaction vessel is transferred to the boiler to fuel the boiler.

31. The photosynthetic system of claim 20, further comprising:
a first solar reflector positioned within the first reaction vessel such that irradiated light that is not absorbed by the first oriented photoactive material is reflected back into the first reaction vessel; and
a second solar reflector positioned within the second reaction vessel such that irradiated light that is not absorbed by the second oriented photoactive material is reflected back into the second reaction vessel.

32. The photosynthetic system of claim 20, wherein at least a portion of the first reaction vessel and at least a portion of the second reaction vessel are formed of a transparent material.
